(19) United States
(12) Patent Application Publication
(10) Pub. No.: US 2005/0080469 A1
(43) Pub. Date: Apr. 14, 2005

(54) Treatment of cardiac arrhythmia utilizing ultrasound

(76) Inventors: Eugene A. Larson, Lummi Island, WA (US); Perry W. Kaminski, Stehekin, WA (US)

Correspondence Address: Robert L. McDowell, 1170 Jackson Heights Drive, Webster, NY, 14580, US

(21) Appl. No.: 10/921,715

(22) Filed: Aug. 19, 2004

Related U.S. Application Data
(60) Provisional application No. 60/500,067, filed on Sep. 4, 2003.
(60) Provisional application No. 60/560,089, filed on Apr. 7, 2004.

(51) Int. Cl. A61F 2/00

(52) U.S. Cl. 607/101

(57) ABSTRACT

A noninvasive or minimally invasive treatment of cardiac arrhythmia such as supraventricular and ventricular arrhythmias, specifically atrial fibrillation and ventricular tachycardia, by treating the tissue with heat produced by ultrasound, (including High Intensity Focused Ultrasound or HIFU) intended to have a biological and/or therapeutic effect, so as to interrupt or remodel the electrical substrate in the tissue area that supports arrhythmia.

This application claims the benefit of U.S. Provisional Patent Application No. 60/500,067 filed Sep. 4, 2003 and U.S. Provisional Patent Application No. 60/560,089 filed Apr. 7, 2004.

### FIELD OF THE INVENTION

The present invention is directed to the noninvasive or minimally invasive treatment of cardiac arrhythmias such as supraventricular and ventricular arrhythmias

### BACKGROUND OF THE INVENTION

In the United States, an estimated 2.5-3.0 million individuals experience clinically significant supraventricular and ventricular arrhythmias each year. There is a prevalence of over 2,000,000 and 500,000 new cases annually of atrial fibrillation (AF) and flutter respectively in the United States. Atrial fibrillation is believed to be responsible for 75,000 ischemic strokes at a projected cost of 44 billion dollars annually in the United States. Approximately 8% of those over 65 suffer from atrial arrhythmia. Each year, AF is responsible for over 200,000 hospital admissions and 1.5 million outpatient visits and procedures. Ventricular tachycardia afflicts about 400,000 people annually in the United States. Developed countries worldwide with Western profiles of heart disease experience similar prevalence. More than 1 million electrophysiology procedures (EP) are performed annually worldwide for the treatment of arrhythmias. The approximate cost of an EP treatment for arrhythmia in the US is $16,000.

Atrial fibrillation and atrial flutter are the most common arrhythmias encountered clinically. Current strategies for treating these arrhythmias include drugs used for rate control, maintenance of sinus rhythm, and stroke prevention. Recently there has been an enthusiasm for nonpharmacologic options for the treatment of atrial fibrillation and atrial flutter. This enthusiasm has been driven by the poor efficacy of drugs for maintaining sinus rhythm long term and the significant side effects associated with many of these medications. Some of these nonpharmacologic treatment options available for treating atrial fibrillation and flutter include:

Radio frequency ablation of atrial flutter targeting the “isthmus” of tissue between the tricuspid valve and inferior vena cava.

Implantation of an atrial defibrillator.

Radio frequency ablation of the atrio—ventricular node followed by implantation of a pacemaker.

Surgical “maze” procedure requiring an open thoracotomy and in most cases cardiopulmonary bypass

Catheter based pulmonary vein isolation procedures during which the pulmonary veins are isolated segmentally or circumferential pulmonary vein ablation strategies aimed at remodeling the posterior left atrium, an important substrate for the propagation of atrial fibrillation.

These therapies have morbidity and mortality liabilities, including:

1. The risk of stroke and air-embolization associated with moving catheters in the left atrium.

2. Significant procedure duration owed to the technical difficulties in accomplishing pulmonary vein isolation.

3. Cardiac perforation from roving mapping and ablation catheters within the thin walls of the left atrium while the patient is fully anticoagulated.

4. Esophageal injury.

5. Pulmonary vein stenosis.

6. Bleeding, patient discomfort and pain, infection, precipitation of heart failure, and long hospital stays associated with cardiothoracic surgery in the case of the “maze” procedure.

### SUMMARY OF THE INVENTION

The present invention is directed to the noninvasive or minimally invasive treatment of cardiac arrhythmia such as supraventricular and ventricular arrhythmias, specifically atrial fibrillation, atrial flutter and ventricular tachycardia, by treating the tissue with heat produced by ultrasound, (including High Intensity Focused Ultrasound or HIFU) intended to have a biological and/or therapeutic effect, so as to interrupt or remodel the electrical substrate in the tissue area that supports arrhythmia.

### BRIEF DESCRIPTION OF THE DRAWINGS

FIG. 1 shows a lesion produced intraoperatively in the posterior wall of an animal heart.

FIGS. 2A and 2B are photographs of sub-lethal damage to arterial wall tissue produced by relatively low levels of HIFU.

FIGS. 3A, 3B and 3C illustrate, respectively, linear, spherical, and sectioned annular phased arrays of ultrasound transducers.

FIGS. 4A and 4B show field distributions of, respectively, time averaged intensity and heat rate of a 20 element sectioned annular phased array.

FIGS. 5A, 5C and 5E show temperature evolution at different time intervals while FIGS. 5B, 5D and 5F show respective lesion formation due to HIFU exposure for the model shown in FIGS. 2A and 2B.

FIGS. 6A and 6C show temperature evolution at different time intervals while FIGS. 6B and 6D show respective lesion formation due to continuous HIFU exposure for the model shown in FIGS. 2A and 2B.

### DETAILED DESCRIPTION OF THE PREFERRED EMBODIMENT

The development of interstitial fibrosis and electrophysiological changes including a decrease in the number and distribution of gap junctions within the atria, shortening of atrial refractory periods, and a dispersion of refractoriness, lend to the substrate factors promoting the propagation of atrial fibrillation.

The atrial remodeling may be secondary to other cardiac structural disorders such as valvular heart disease, rheumatic heart disease, coronary artery disease, or viral myocarditis but may also occur as a result of clinical exposure to the arrhythmia. Significant electrical and structural remodeling is known to occur in patients with otherwise normal hearts who have been exposed to long periods of atrial fibrillation.

Triggers of atrial fibrillation may be due to ectopic atrial foci (usually from the pulmonary veins), atrial flutter, or other supraventricular arrhythmias. In patients with structurally normal hearts, ectopic foci from the pulmonary veins are known to serve as triggers of atrial fibrillation in greater than 95% of patients. Primary drivers in the electrically active sleeves of myocardial tissue within the pulmonary veins serve as either the triggers for, or the maintenance of, atrial fibrillation. The drivers also may originate in the superior vena cava, ligament of marshal, coronary sinus and other sites within the left and right atrium. Secondary drivers may form in response to the primary drivers and perpetuate atrial fibrillation. Short cycle wavelengths form rotors which have anchor points near the pulmonary veins. Termination of atrial fibrillation is accomplished by eliminating the primary and secondary drivers or eliminating the anchor points of the rotors. In the case of multiple wavelet reentry as a perpetuation of atrial fibrillation, modification of the atrial substrate can prevent these wavelets from developing.

Persistent atrial fibrillation develops as the atrial substrate continues to remodel (fibrosis, enlargement, changes in electrophysiology) from increasing exposure to atrial fibrillation and to the hemodynamic consequences of atrial fibrillation. The likelihood of persistent atrial fibrillation is augmented by the presence of structural heart disease (congestive heart failure, valvular heart disease, etc.).

Ventricular tachycardia may result from a number of mechanisms. Most ventricular tachycardias are encountered in patients with ischemic cardiomyopathy and are due to reentry. Focal sources of ventricular tachycardia occur due to increased autonomaticity or triggered activity. In patients with structural heart disease, most symptomatic ventricular arrhythmias are mediated by re-entry within the transitional zone between scar and healthy myocardium. In patients without structural heart disease, ventricular arrhythmias often originate in the right ventricle outflow track or in the purkinje network of the conduction system (idiopathic left ventricular tachycardia). Currently, catheter based strategies for mapping and ablation of ventricular tachycardia is accomplished with reasonable success rates with catheter based delivery of RF energy applied to the site of origin of focal ventricular tachycardia or at the vulnerable limb of the reentry circuit in the case of ischemic ventricular tachycardia. HIFU can be a preferred energy source for the treatment of ventricular tachycardia because it can be delivered less invasively and may be focused endocardially or epicardially.

The present invention describes the creation of controlled transmural lesions, or, accelerated cell apoptosis and local collagen or cellular reconfiguration, accomplished by sublethal cellular heating, which remodels electrical conduction. Ablation and cell apoptosis occurs at about 60° C. or above; structural protein remodeling, changes in the shape of protein and phase transition occur between about 50° C. and about 60° C.; and at about 40° C. or below, no permanent cellular changes or damage occurs. This therapeutic approach results in ablation of arrhythmia and can also induce regeneration of normally functioning cardiac tissue.

An in vivo animal experiment was designed and carried out to demonstrate the effectiveness of producing an acoustocautery lesion using High Intensity Focused Ultrasound (HIFU) in a live pig heart. The goal was to produce a lesion in the endocardium of the posterior left ventricular wall by applying HIFU intraoperatively through the heart from the outside surface of the anterior left ventricular wall. The unfocused HIFU energy passed first through the anterior myocardium of the left ventricle, then through the blood-filled ventricular chamber to reach the endocardium of the posterior left ventricular wall where the HIFU power was focused. Tissue within the focal region, where the spatial peak intensity was greatest, was heated due to absorbed energy creating a lesion.

For this study, a HIFU system was utilized with total forward electrical power set to 60 watts. A HIFU transducer was selected with 4 MHz center frequency and 5 cm focal length. Because the region of interest in the myocardium was less than 5 cm from the front face of the transducer a truncated hydrogel cone was placed between the transducer and the epicardium to serve as an acoustic standoff. Hydrogel was chosen as the acoustic coupling path within the standoff because it is easy to handle and it is relatively unattenuating to the unfocused ultrasound energy propagating through it.

The transducer with truncated conical standoff was placed on the anterior left ventricular wall of the beating heart and acoustic power applied in a single burst of ten seconds. Ultrasound energy generated within the transducer passed through the hydrogel, the anterior wall of the heart, the blood-filled ventricle, and focused on the endocardium of back wall of the left ventricle.

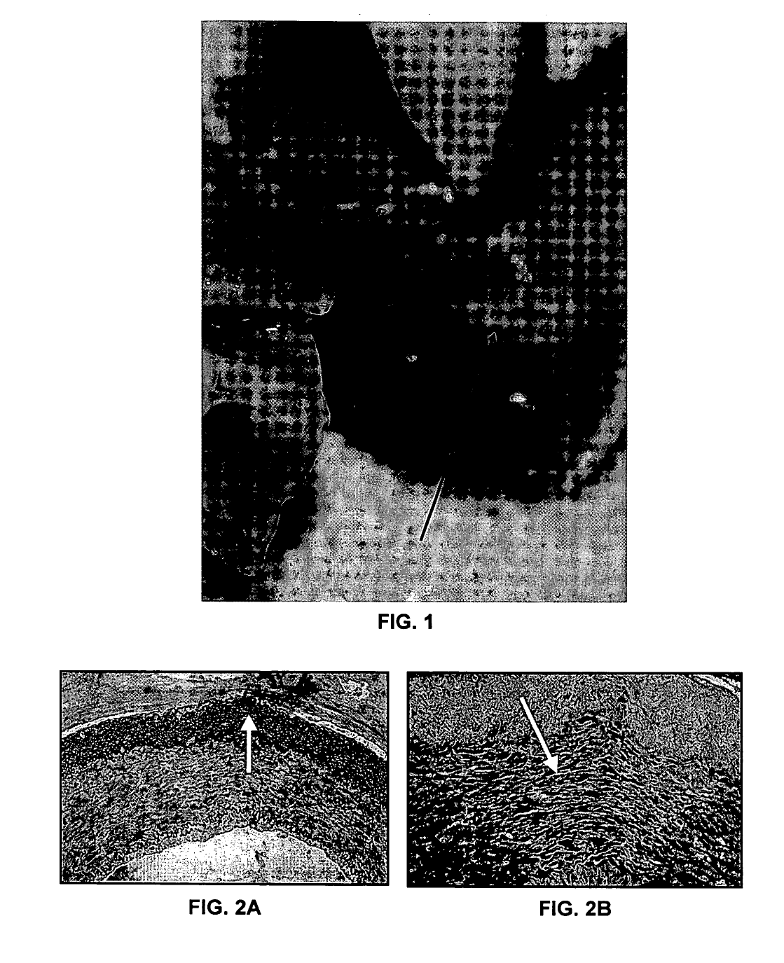

A lesion on the posterior ventricular myocardium was successfully created using HIFU applied from the anterior wall through the left ventricular cavity to the posterior wall. The photograph in FIG. 1 shows the lesion produced intraoperatively in the posterior wall with the transducer device placed on the epicardium of the anterior left ventricular wall. The transducer and the origin of the HIFU are to the right of this picture. HIFU energy passed through the anterior wall, the blood-filled ventricular chamber and focused on the endocardium of the opposite posterior left ventricular wall as indicated in this picture. Intervening tissue (the anterior wall) appeared undamaged.

FIGS. 2A and 2B are photographs of sub-lethal damage to arterial wall tissue produced by relatively low levels of HIFU. In FIG. 2A the arrow points to a layer of tissue stained by a Van Gleason stain to show elastin fibers. Note the disruption in the layer. Similarly, FIG. 2B shows tissue stained by a trichrome stain to show collagen fibers. Note the obvious disruption in the fibers. In both cases, the damage produced to these tissues is sub-lethal and will be structurally repaired by the body. It is during this structural repair that electrical normality will be resumed. The arrow in FIG. 2A shows that the elastin fibers (stained black) are damaged, and disrupted. FIG. 2B shows a higher magnification of the area shown in FIG. 2A, and shows that the colligen fibers (stained blue, and indicated by the arrow), located distal to the elastin fibers, are also damaged, although not lethally.

The present invention provides a method for reducing or eliminating arrhythmias within a heart. The method comprises targeting a region of interest of the heart, such as with diagnostic ultrasound or fast computed tomography (CT), emitting therapeutic ultrasound energy from an ultrasound radiating surface, focusing the emitted therapeutic ultrasound energy on the region of interest and, producing sub-lethal or lethal tissue damage in the region of interest of the heart, such as, the atrial wall, the ventricular wall, the inteventricular septum, or any other location within the heart.

Preferably, the inventive method achieves the interrupted or remodeled electrical conduction by steps which include:

(a) ultrasound imaging the area of therapeutic interest of the heart and/or the attached vessels;

(b) gating the tissue/blood interface so as to allow the delivery of High Intensity Focused Ultrasound (HIFU) continuously to the moving interface; and,

(c) delivering ultrasound to or near the point of arrhythmia origin (the primary or secondary drivers), or in the pathway of the arrhythmia (short cycle rotors which have anchor points) with an ultrasound device to induce a controlled amount of cellular damage to a localized area of the heart and/or the attached vessels.

Most preferably, the steps of the inventive method include:

1. Imaging of the heart and specifically the area of therapeutic interest by two or three dimensional Transesophageal Echocardiography or Transthoracic Ultrasound using phased or annular array imaging.

2. Gating of the endocardium (endothelium and subendothelial connective tissue) at the tissue/blood interface to dynamically focus the same or another single or multiple annular or phased array transducer (in the frequency range of 1 to 7 MHz) so as to deliver ultrasound continuously to the moving interface. For example, gating of the endocardium/blood interface may be implemented as follows:

a. The operator of the system identifies the endocardium/blood interface from a one-dimensional m-mode (selected from an array) and positions an electronic “gate” around the excursion of the heart wall.

b. The electronic imaging system (from step 1) tracks the echo within the gate window as it moves axially and generates an analog voltage depth signal.

c. The analog depth signal drives the dynamic focus of the HIFU transducer (changes delay on the fly).

d. Feedback may be provided to the operator by superimposing the HIFU focus on the image.

3. In the case of creating a lesion or destruction of cells where exact acoustic path properties and location are critical, utilizing a micro ultrasound device (combined transmitter and hydrophone transducer) that permits precise location of the electrophysiology mapping catheter and intended therapeutic HIFU focus at the point of the arrhythmia origin or conduction on the ultrasound image (transponder), provides an intracardiac transmit source for phase aberration correction (transmitter), and functions as a hydrophone for confirming the location of the HIFU focus before therapy is initiated.

a. The foci of arrhythmia may be mapped by an EP catheter containing the transponder which functions by ultrasonic wave energy being received by a transducer located on the EP arrhythmia mapping catheter. The received energy is detected and a visual marker is produced on an image display that represents the location of the mapping catheter tip within the heart.

b. The point-source nature of the micro catheter transducer/transponder in (a) above may be utilized with time-reversal algorithms to remove phase aberrations resulting from multiple acoustic paths. Phase aberration correction of the HIFU focus may not be necessary when imaging Transesophageal (TEE), such as for instances of atrial arrhythmia, as the tissue is more uniform than with Transthoracic echocardiography and the atria are in close proximity to the esophagus.

c. The location of the HIFU focus prior to initiating a therapeutic power level may be confirmed by pulsing the HIFU transducer at low power, such as to have no biological effect, and locating the HIFU focus and intensity with the micro catheter transducer/transponder.

4. The directed HIFU acoustic energy and geometric pattern is preferably varied so as to induce cellular damage or change to a specific localized area of the heart and/or the attached vessels. The controlled introduction of cellular damage will result in either rapid and complete necrosis of cells (temperatures of about 60° C. or above) as seen in FIG. 1, partial damage to collagen and muscle fiber tissue as seen in FIGS. 2A or 2B, or changes in the shape of proteins, structural protein remodeling and phase transition (temperatures of about 50° C. to about 60° C.). In either case, tissue regeneration or structural remodeling, resulting from this induced heat from ultrasound, will result in a return to normal electrical conduction characteristics over time, or, the complete or partial interruption of the arrhythmia electrical pathway.

The inventive method thus provides for the non-invasive or minimally invasive treatment of atrial fibrillation, atrial flutter and ventricular tachycardia utilizing HIFU (preferably in the frequency range of 1-7 MHz, but not limited thereto), to:

a. create a well controlled lesion of determinable volume (depth and shape), which neither bleeds, chars nor immediately erodes, to terminate atrial fibrillation, atrial flutter and ventricular tachycardias through interruption of the electrical pathway. In the example of Atrial Fibrillation, this may be accomplished by creating the lesion (ablation) pathway in a manner that encircles the pulmonary veins and/or separates the anchor points of short wavelength drivers.

OR

b. accelerate apoptosis, or cause injury to cardiac cells, or cause phase transition, changes in the shape of cell proteins or structural protein remodeling in a well defined volume, so that they regenerate over time in a predictable manner which restores normal electrical function to cardiac cells which have abnormal conduction or are the focus for arrhythmias. In the case of atrial arrhythmias, this ultrasound generated heat therapy to the atrial substrate can cause disruption or elimination of primary or secondary drivers, disruption of rotors and the critical number of circulating wavelets or the elimination of the rotor anchor points which surround the pulmonary veins. The pathway for cell heat regeneration therapy may encircle the Pulmonary veins and/or include an area of the left and right Atrium thereby disrupting the formation or conduction of short wavelength rotors and their anchor points.

The inventive method is preferably carried out through utilization of the following:

1. Two or three dimensional phased or annular array imaging and gating of the heart endocardium or vessel endothelium through Transesophageal or Transthoracic ultrasound imaging allows for dynamically controlling the therapeutic ultrasound focus in the diseased heart whereas synchronizing to an ECG signal does not represent true heart wall and vessel motion. Transesophageal imaging and HIFU therapy is particularly applicable to arrhythmia originating in the left and right atrium given the proximal location of the esophagus to the atria.

2. Array therapy ultrasound transducers (single or multiple) dynamically focused by a gated signal from ultrasound imaging, as in 1 above. The transducer may be annular or oval arrays or phased array technology in the frequency range of 1-7 MHz. The HIFU therapy transducer can be the same transducer that is used for imaging or a separate transducer used in synchrony with the imaging transducer.

3. In the case of creating a lesion or destruction of cells where exact acoustic path properties and location are critical, an in-dwelling cardiac acoustic transponder/hydrophone/transmitter can be utilized. A thin film plastic or ceramic piezoelectric chip mounted on an electrophysiology mapping catheter lead which:

a. permits location of HIFU transducer focus as well as at the foci or path of cardiac arrhythmia origin or conduction on the ultrasound image.

b. provides a point source ultrasound transmitter from the site of ablation interest back to both the HIFU and the imaging transducer which in turn provides phase aberration correction feedback data for accurately generating the HIFU focus and provides a method for overcoming diffraction limits by expanding the effective aperture of the ultrasound transmitter.

4. The design of a transducer array can take many forms. We provide below some specific approaches to this array design as well as provide some details on the use of this array to produce either lethal or sub-lethal effects in cardiac tissue.

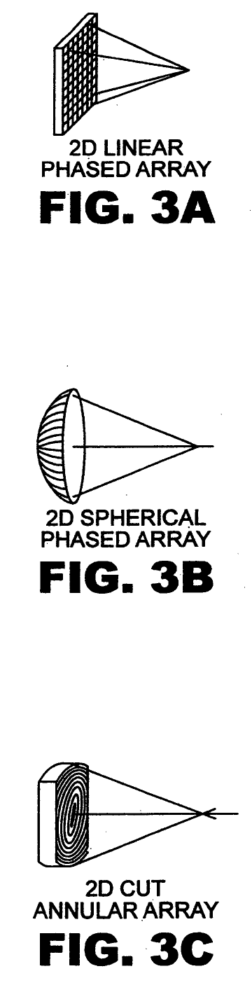

The following HIFU system design can be utilized for either Trans-esophageal or Trans-thoracic treatment of atrial arrhythmia and ventricular tachycardia. In one embodiment, the system is composed of two-dimensional, independent multi-channel-multi-element arrays that will be used in both imaging (low power, high dynamic range) and treatment (high power, low dynamic range) modalities. The ultrasound transducers can be linear, spherical, or sectioned annular phased arrays (as shown in FIGS. 3A, 3B and 3C, respectively), and will operate in the frequency range of 1-7 MHz as to provide good imaging resolution (higher ranges) and sufficient therapeutic focal power deposition (low-middle ranges) without in-path collateral damage.

Linear and spherical phased arrays will provide three degrees of freedom and will allow electronic steering of the focal region in a three-dimensional domain without constraints. Sectioned annular arrays, on the other hand, will only allow electronic dynamic focusing on the propagation axis, in which case the transducer will be mechanically moved (up or down) and rotated on its long symmetry axis to provide complete sweeps of desired volumes. In this particular design, the loss in electronic steering freedom is compensated by a more efficient power transfer and focusing gain with greatly reduced side lobes.

Linear and spherical phased arrays are the preferred designs for external, transthoracic applications. In this approach, the strongly inhomogeneous nature of the intervening tissue between the transducer and the atrium requires maximum flexibility in the array phasing for accurate targeting and for minimizing phase aberrations that would significantly deteriorate the focal characteristics. Furthermore, because there are no major restrictions on the size of the HIFU system, a wide aperture and a large number of elements can be used to assure desired power deposition at deeper focal positions.

Conversely, given the limited circular dimension of the esophagus (circa 1.5 cm), and the close proximity of the left atrium, for trans-esophageal applications, small (e.g. 1.1 cm in width, 0.7 cm in depth, and 4-6 cm in elevation) linear or sectioned annular arrays will be the preferred embodiment.

Targeting of the region of interest (ROI) in the diseased heart can be performed either statically or dynamically:

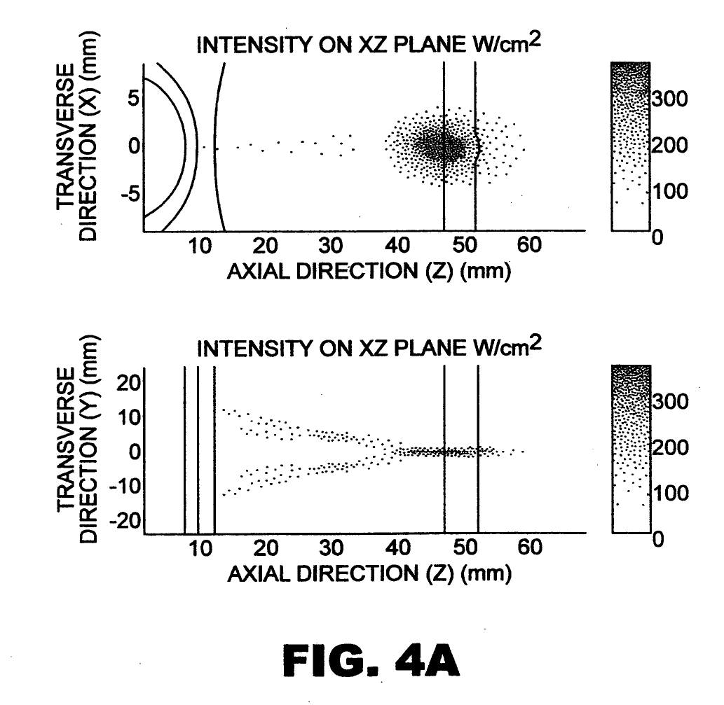

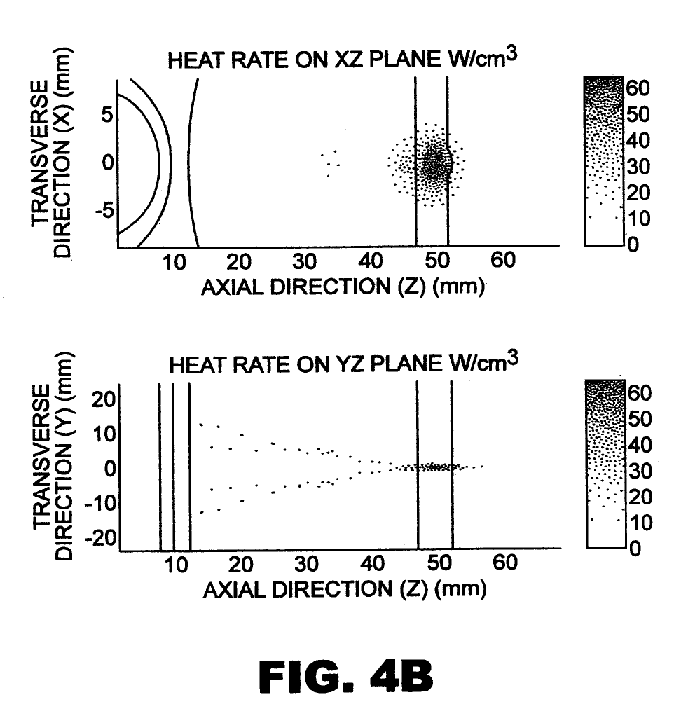

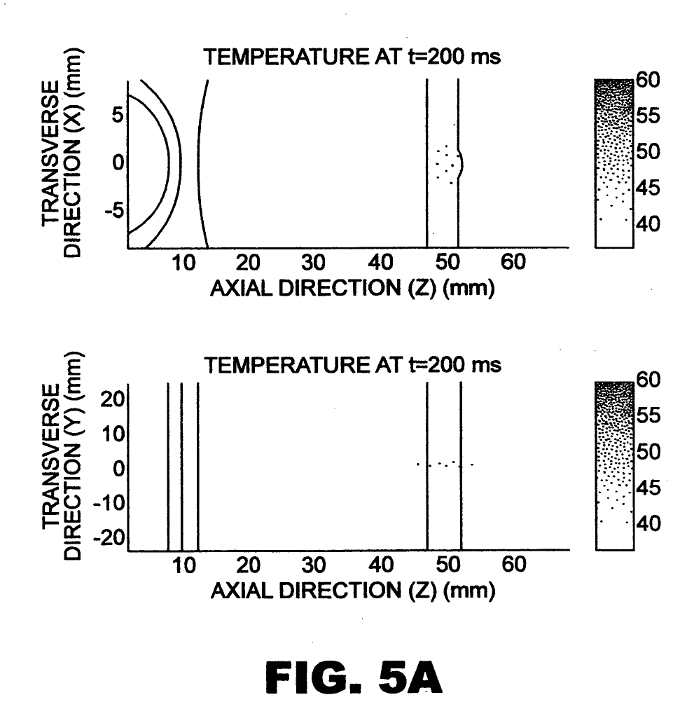

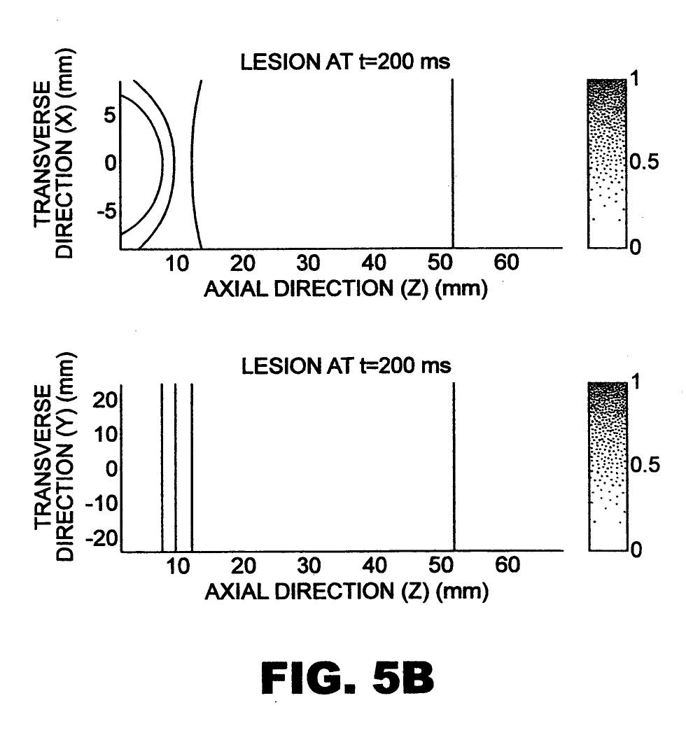

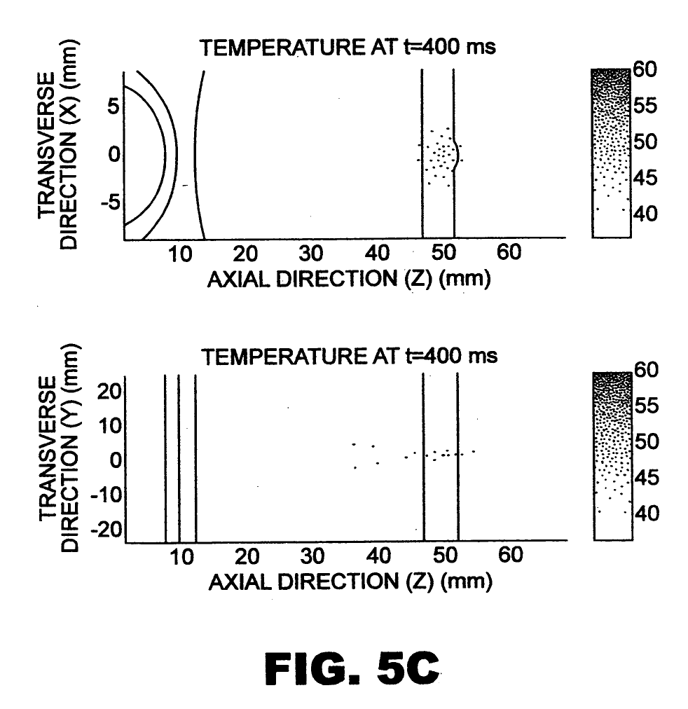

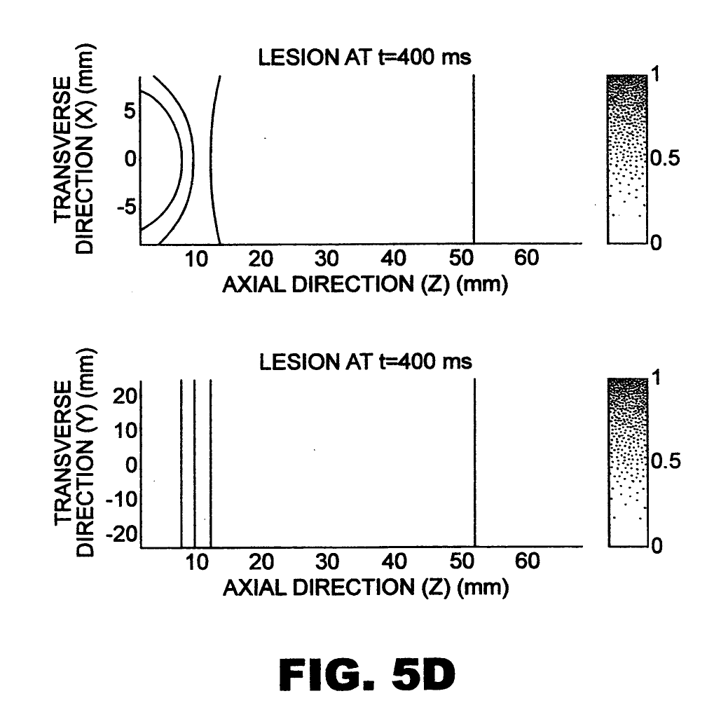

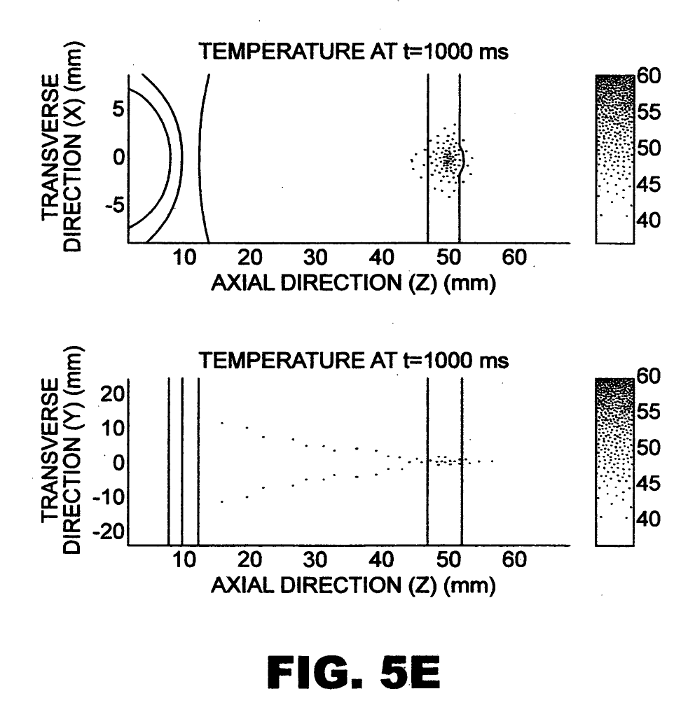

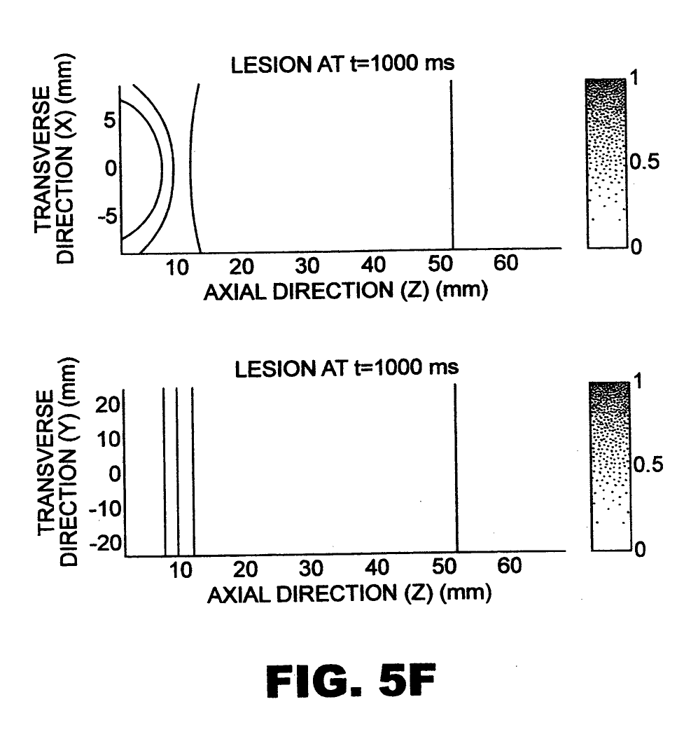

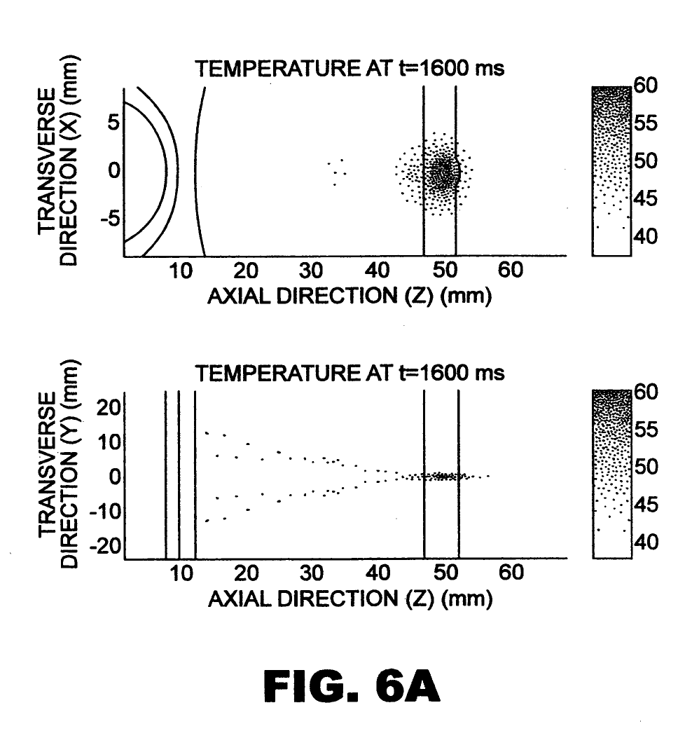

Static targeting: In this embodiment, the ROI is initially imaged in B-mode, the position of the endocardium/blood interface is acquired from the image (pulse-echo time of flight), and the HIFU system is properly phased to focus on this target. The HIFU system is phased-locked with an electrocardiogram (ECG) and therapy delivered only at diastole when the heart boundary is in the focal zone of the transducer. Drugs such as beta-Adrenergic Blockers can be used to reduce the heart rate and will provide approximately 0.3 seconds of diastolic time. This time frame is more than enough to induce temperature increases in cardiac tissue of the order of 15 to 25 degrees Celsius depending on the acoustic power applied (see FIGS. 4 and 5, discussed below, for example).

FIG. 4 shows field distributions of time averaged intensity (FIG. 4A) and heat rate (FIG. 4B) of a 20 element sectioned annular phased array, similar to that shown above in FIG. 3C, for transesophageal acoustic propagation in a model of the heart and focusing on the distal heart wall. The HIFU system is located on the left inside the esophagus. The tissue layers correspond to esophagus, proximal heart wall, blood, distal heart wall, and fluid.

FIGS. 5A, 5C and 5E show temperature evolution at different time intervals while FIGS. 5B, 5D and 5F show respective lesion formation (defined by the thermal dose criterion common to thermal therapy) due to gated HIFU exposure for the model shown in FIGS. 2A and 2B. Note that lethal lesion formation is prevented, the goal of this particular modality. For this computation, the HIFU is assumed to be applied only during a 0.3 second interval associated with diastole, in which the heart tissue is assumed to be stationary. In this case, the applied HIFU therapy results in heating of the tissue to temperatures in excess of 45° C., but as shown in FIGS. 5B, 5D and 5F, with insufficient thermal dose to result in tissue necrosis. Thus, this case as shown in FIG. 5 results in a non-lethal HIFU dose.

Dynamic targeting: dynamic targeting can be accomplished in two ways. The first approach is based on the method described earlier for static targeting. In this case, an electronic gate around the excursion of the heart wall is determined from acquired B-mode images. The system (in imaging mode) will track the endocardium/blood interface echo within this gate as it moves axially and will generate a depth signal which will drive the HIFU transducer (in therapy mode) with the proper delays to move the focus accordingly to the heart motion.

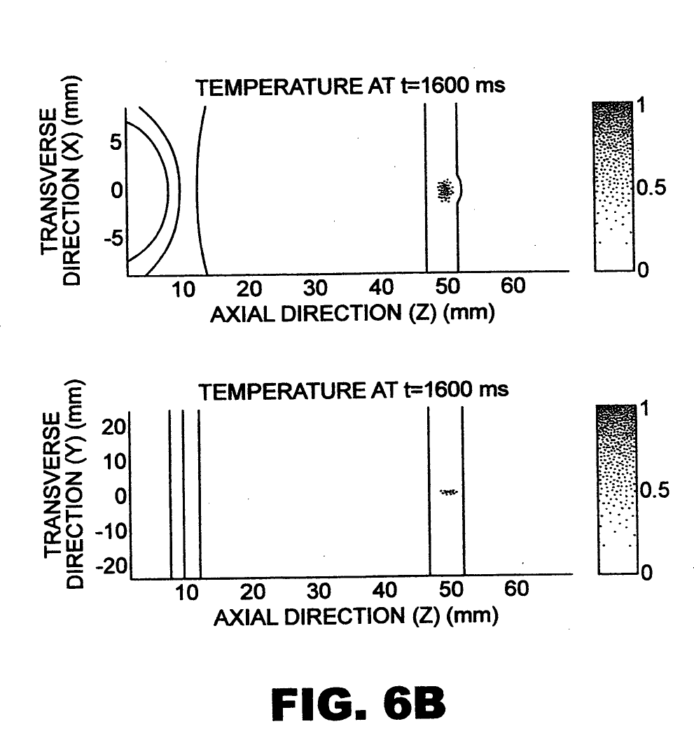

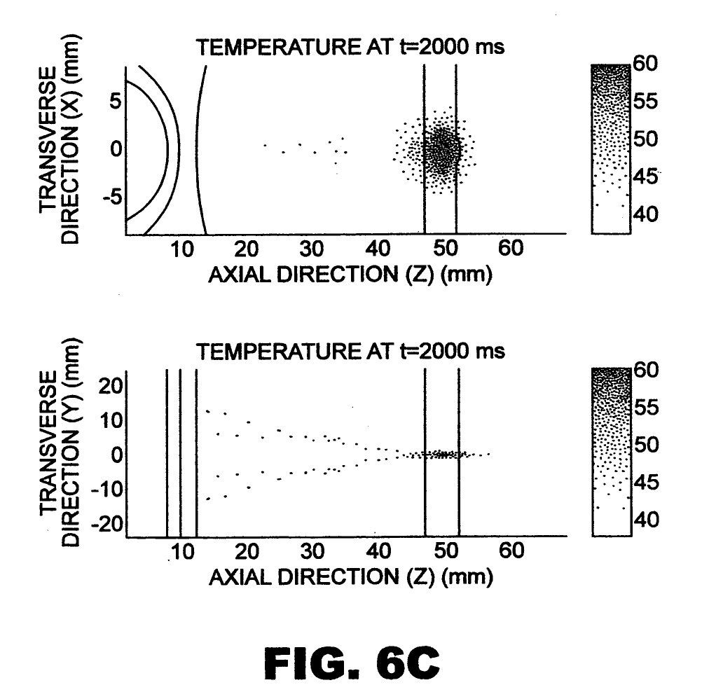

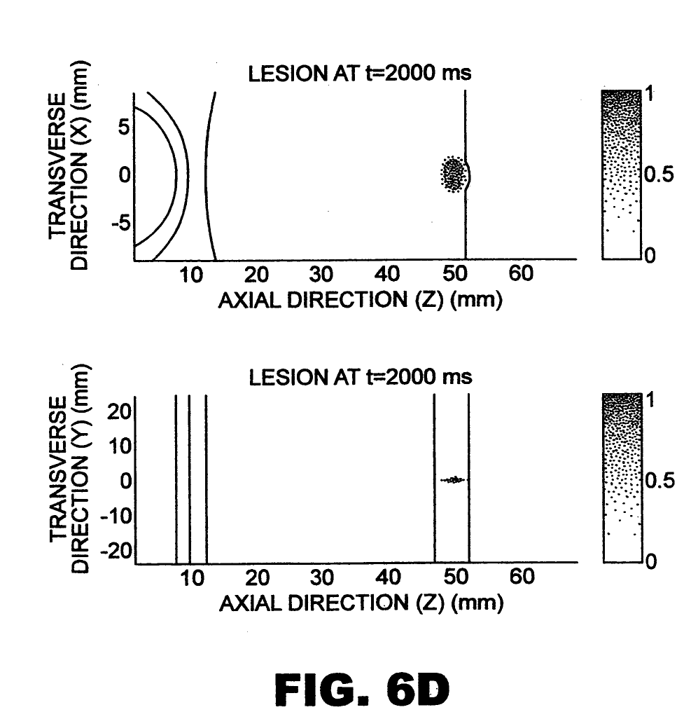

The second approach of dynamic targeting involves the use of a micro ultrasonic device (transponder) mounted on an electro-physiology mapping catheter. The transponder will generate a source signal received by the therapy array and utilized with time-reversal algorithms to dynamically correct for phase aberrations resulting from multiple acoustic paths and compensate for the target motion. In this fashion, the focal region of the system will be able to continuously track the same target region as it moves. In this case, HIFU can be applied continuously and lethal tissue damage can be obtained (see FIG. 6, for example). FIGS. 6A and 6C show temperature evolution at different time intervals while FIGS. 6B and 6D show respective lesion (thermal dose criterion) formation due to continuous HIFU exposure for the model shown in FIGS. 2A and 2B. In this example, lesion formation is desired, and occurs exclusively into the endocardium due to the low absorption of both blood and external fluid. For this computation, the HIFU is assumed to be applied only during a 0.3 second interval associated with diastole, in which the heart tissue is assumed to be stationary. The applied HIFU therapy results in heating of the tissue to temperatures in excess of 65° C., and as shown in FIGS. 6B and 6D, with sufficient thermal dose to result in tissue necrosis. Thus, the case as shown in FIG. 6 results in a lethal HIFU dose.

The multi-element designs of the HIFU system provide flexibility in terms of focal spot dimensions. By properly choosing the individual phases and time delays of each element in the array, the focal dimensions and characteristics of the system can be manipulated from a high-power small, grain-of-rice-size focus, to a low-power large, navy-bean-size focal volume. For example, with an acoustic intensity on the order of 2 kW/cm² and a driving frequency of 2 MHz, tissue temperatures can be elevated to 100° C., from an ambient level of 37° C., within a few seconds. Modeling as illustrated in FIGS. 4, 5 and 6 accounts for nonlinear effects, tissue perfusion, temperature and frequency dependent absorption. Therefore, predicted temperatures can be as accurate to within a few degrees Celsius. With this level of control, it is possible to produce either sub-lethal or lethal tissue damage, with either a trans-esophageal or a trans-thoracic approach.

One of the strengths of HIFU over competing ablation technologies is the superior control that is available to the user, and this control takes many forms For example, because the focal volume of the therapy transducer is normally quite small (varying from a grain of rice to a navy bean in size), one has relatively precise control over the spatial extend of the tissue lesion that is produced. In addition, because the temperature elevation is so rapid (50 degrees Celsius per second, for example), blood perfusion does not affect the shape of the lesion, and its shape and size can be reliably repeated. Finally, because the duration of the applied HIFU can be controlled so precisely (to within a few acoustic cycles at 2 MHz), local tissue temperatures can be controlled to within a few degrees Celsius. This temperature control allows one to selectively treat different tissue types. For example, muscle tissue can be necrosed but the vasculature remains intact, due to the cooling effect of blood within the vessels. In addition, connective tissues are more capable of withstanding elevated temperatures than muscle cells, and thus, with proper control of the local tissue temperature, myocardial tissues can be necrosed without damage to the surrounding matrix of connective tissues.

Depending on the application, whether for complete cellular necrosis or structural protein remodeling, one approach will be more effective than the other, even though, in both applications, the treatment volume is usually larger than the transducer's focal area. Large volume treatments can be performed following two different approaches: (1) by discrete-step steering of the transducer focus, in which treatment is discretely delivered at adjacent locations in the volume, or (2) by continuous steering where the volume is uninterruptedly treated in a “painting”-type fashion.

In some arrhythmias, the region of arrhythmia origin can be located by external mapping utilizing triangulation or vectoring. These arrhythmias may be able to be treated with levels of therapeutic ultrasound that cause electrical remodeling with or without local but controlled cell apoptosis.

The present invention provides patient benefits which include:

1. a unique, durable non-invasive or minimally invasive therapeutic approach directly to the beating heart for the treatment of cardiac arrhythmias, most commonly atrial fibrillation, atrial flutter and ventricular tachycardia.

2. the elimination of pulmonary vein stenosis in the treatment of atrial fibrillation.

3. the reduction or elimination of the associated morbidity and mortality from competing procedures, such as bleeding, blood clots, potential for stroke and pulmonary embolism.

4. the ability to repeat the therapeutic ultrasound arrhythmia ablation procedure indefinitely with only minor morbidity.

While the invention has been described with reference to preferred embodiments it is to be understood that the invention is not limited to the particulars thereof. The present invention is intended to include modifications which would be apparent to those skilled in the art to which the subject matter pertains without deviating from the spirit and scope of the appended claims.

1. A method for reducing or eliminating arrhythmias within a heart, said method comprising:
targeting a region of interest of the heart by diagnostic imaging;
emitting therapeutic ultrasound energy from an ultrasound radiating surface placed non-invasive on the skin or minimally invasive in the esophagus;
focusing the emitted therapeutic ultrasound energy on the region of interest; and,
producing sub-lethal or lethal tissue or cellular damage in the region of interest.

2. The method of claim 1 wherein said targeting is carried out with diagnostic ultrasound.

3. The method of claim 1 wherein the region of interest comprises an atrial wall.

4. The method of claim 1 wherein the region of interest comprises a ventricular wall or interventricular septum of the heart.

5. The method of claim 1 wherein the damage to the region of interest is lethal tissue or cellular damage.

6. The method of claim 1 in which the ultrasound radiating surface is located in the esophagus.

7. The method of claim 1 in which the ultrasound radiating surface is located on the skin and the energy is delivered transthoracically.

8. The method of claim 7 wherein the energy is delivered intercostally or subcostally.

9. The method of claim 1 in which the emitted ultrasound energy is gated by an ECG to deliver energy during a limited period of heart wall motion.

10. The method of claim 2 in which pulse echo signals from the diagnostic array is used to deliver the emitted therapeutic ultrasound energy in phase with the heart motion thereby delivering ultrasound energy continuously.

11. The method of claim 3 in which the emitted ultrasound energy produces sub-lethal tissue damage in a region of at least one of the left and right atrium thereby altering electrical conduction.

12. The method of claim 4 in which the emitted ultrasound energy produces sub-lethal tissue damage in a region of at least one of the left and right ventricular wall or interventricular septum thereby altering electrical conduction.

13. The method of claim 2 in which the emitted ultrasound energy produces lethal tissue damage to predetermined regions in the heart thereby causing at least one of disruption to the primary or secondary drivers, disruption of rotors and the critical number of circulating wavelets, and the elimination of the rotor anchor points which surround the pulmonary veins.

14. The method of claim 1 in which the emitted ultrasound energy produces sub-lethal tissue damage to previously determined regions in the heart, promoting at least one of tissue and cellular changes which results in the reduction of cardiac arrhythmias.

15. The method of claim 1 wherein the arrhythmias comprise at least one of atrial arrhythmia and ventricular arrhythmia.

16. The method of claim 15 wherein said atrial arrhythmia comprises atrial fibrillation and/or atrial flutter.

17. The method of claim 15 wherein said ventricular arrhythmia comprises ventricular tachycardia or frequent premature ventricular contractions.

18. The method of claim 1 wherein said therapeutic ultrasound comprises high intensity focused ultrasound.

19. The method of claim 2 wherein said targeting further comprises:
placing an ultrasonic device at the region of interest, said device generating a signal which identifies the origin of arrhythmia via the diagnostic imaging and provides a focus location for the therapeutic ultrasound, and wherein the device signal is also received by the imaging transducer and electronics which provide phase aberration correction feedback data to the therapeutic ultrasound system to accurately generate the therapeutic ultrasound focus and to overcome diffraction limits by expanding the effective aperture of the therapeutic ultrasound transducer.

20. A method for providing for non-invasive or minimally invasive treatment of atrial arrhythmia and ventricular arrhythmia utilizing therapeutic ultrasound, said method comprising:
creating a controlled lesion of predetermined depth and shape to terminate atrial and/or ventricular arrhythmias through interruption or changes to the electrical pathway, or the acceleration of apoptosis, at a predetermined region of interest
or,
inducing at least one of injury to cardiac cells, phase transitions, changes in the shape of cell proteins, and structural protein remodeling in a defined volume, whereby the tissues regenerate over time in a manner which reduces, eliminates or prevents the development of cardiac arrhythmias.
